(19) United States
(12) Patent Application Publication
(10) Pub. No.: US 2005/0276835 A1
(43) Pub. Date: Dec. 15, 2005

(54) Photo-reactive polymer platform for use in biomedical devices

(76) Inventors: Joerg Lahann, Ann Arbor, MI (US); Robert S. Langer, Newton, MA (US)

Correspondence Address: Matthew E. Connors;Gauthier & Connors LLP, Suite 2300, 225 Franklin Street, Boston, MA, 02110, US

(21) Appl. No.: 11/119,645

(22) Filed: May 2, 2005

Related U.S. Application Data
(60) Provisional application No. 60/567,278, filed on Apr. 30, 2004.

(51) Int. Cl. A61F 2/06; A61L 2/00

(52) U.S. Cl. 424/422; 424/423; 427/2.24; 528/220

(57) ABSTRACT

A polymer having the following structure: 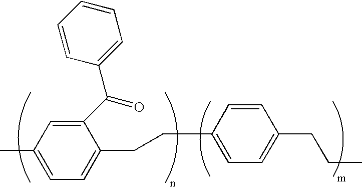 where m is 0 to 10000 and n is 0 to 10000. The polymer may be used in a coating such as on a biomedical device. The polymer is an adhesion promotor for a drug delivery system wherein the polymer is coated onto a substrate such that it may be a reactive coating for UV crosslinking. The polymeric coating may also provide an interface for an implant and can be modified with a secondary polymer or polymer combination. The secondary polymer is capable of eluding drugs or encapsulating cells.

This application claims priority to U.S. Provisional Patent Application No. 60/567,278, filed Apr. 30, 2004. This application is incorporated herein by reference in its entirety.

### BACKGROUND OF THE INVENTION

Percutaneous transluminal coronary angioplasty (PCTA) is a medical procedure, in which a small balloon-tipped catheter is passed through a narrowed coronary artery and then expanded to re-open the artery. Gradual re-narrowing of an artherosclerotic coronary artery, known as restenosis, occurs in 10-50% of patients undergoing PCTA and subsequently requires either further angioplasty or coronary artery bypass graft. 30% of patients with subtotal lesions and 50% of patients with chronic total lesions will go on to restenosis after angioplasty. Thus, successful prevention of restenosis could result not only in significant therapeutic benefit but also in significant health care savings. Vascular stents have been used clinically for more than a decade to treat peripheral arterial occlusive disease percutaneously. In the early years, the scientific interest focused on improvements in the basic characteristics, such as metallurgy and wire mesh design, of metal stents. Since restenosis could not be controlled with simple design modifications, coated stents are believed to be the devices of the future. Several methods and types of coatings were used in the past, and these coatings ranged from inorganic to organic, biodegradable to non-biodegradable, and naturally occurring to synthetic materials. Recently, promising clinical results were presented for drug-eluting stents releasing Paclitaxel. Paclitaxel is an anti-proliferative and anti-inflammatory molecule tested in a series of clinical trials called Taxus. The recently published data of the TAXUS IV randomized trial show extremely low restenosis rates after 9 months. The long-term effects of drug-polymer formulation are unknown. Besides that, stent-based SRL delivery may delay maturation and normal endothelial function, which increases the potential for late thrombotic events. Early clinical results also indicate the risk of sub-acute thromboses (SAT) and hypersensitivity reactions associated with use of the Cordis CYPHER™ stent.

In spite of great progress, the efficacy of drug-eluting stents for the prevention of restenosis has been limited by polymer biocompatibility, suitability for drug combinations, pharmacokinetic properties, and local drug toxicity. This fuels the continuous need for advanced polymer technology fabricating effective and biocompatible drug carriers.

Patterned polymer coatings have attracted attention as elements in biosensors, actuators and have found uses in drug delivery and cell patterning. Spatial definition of secondary polymers is typically achieved via photolithography. More recently, capillary force lithography (CFL) was introduced as an alternative because it is believed to improve the spatial resolution of secondary polymer elements. One advantage of this technique as compared to photolithography is that conventional masks often fail to fabricate features below 10 μm and expensive equipment is needed to obtain pattern fidelity.

The ability to prepare small and discontinuous features is limited to poor adhesion of secondary polymer elements. To overcome this challenge, sophisticated surface engineering techniques must be developed that permit I) high-resolution definition, ii) stable attachment of integrated secondary polymers, and iii) compatibility with microfabrication processes and materials used. For a small class of commonly used process materials (gold, glass, silicon), surface protocols-mainly based on self-assembled monolayers (SAMs) were worked out to prepare confined secondary polymer elements. Nonetheless, generically applicable surface modification protocols are essential to widen the applicability of polymer-integrated microdevices.

Reactive polymer coatings that can be deposited as convergent films on virtually any substrate material provide a flexible solution to surface engineering challenges as they decouple surface design from bulk properties. Consequently, reactive polymer coatings have the potential to provide interfaces for implants to be modified with a secondary polymer or polymer combination. Secondary polymers typically will add an additional function to the implants, such as the capability to elude drugs or to encapsulate cells, including genetically modified cells for gene therapy.

### SUMMARY

A polymer that can act as an adhesion promoter for the drug delivery system and at the same time provides reactive coatings for UV crosslinking, which will hold the drug delivery polymer in place. Thereby eliminating one process step. In addition, the portion of deactivated drug due to crosslinking is minimized with the matrix since the reactive groups are only at the surface and not mixed into the drug delivery polymer. Moreover, patterned substrates can be fabricated accoding to the invention. The use of a photoactive adhesion film with the photoreactive group being a part of the polymer backbone is based on a novel polymer. Due to the use of such a polymer, both the conceptual approach and the materials and matter aspect of the technology is novel and should compete with the existing state-of-the-art technologies.

The polymer can be used to coat biomedical devices, for example stents, with secondary polymers, such as drug eluding polymers.

An object of the present invention is to provide a polymer having the following formulation: 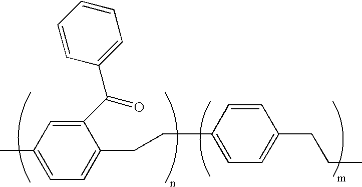
where m is 0 to 10000 and n is 0 to 10000.

### DESCRIPTION OF THE DRAWINGS

Other objects, features and advantages of the present invention will become more apparent as the description proceeds with reference to the accompanying drawings, wherein:

FIG. 1 is a chemical scheme;

FIG. 2 is a schematic of the stabilization of secondary polymers against water; and

FIGS. 3a-e are structures fabricated on reactive coatings.

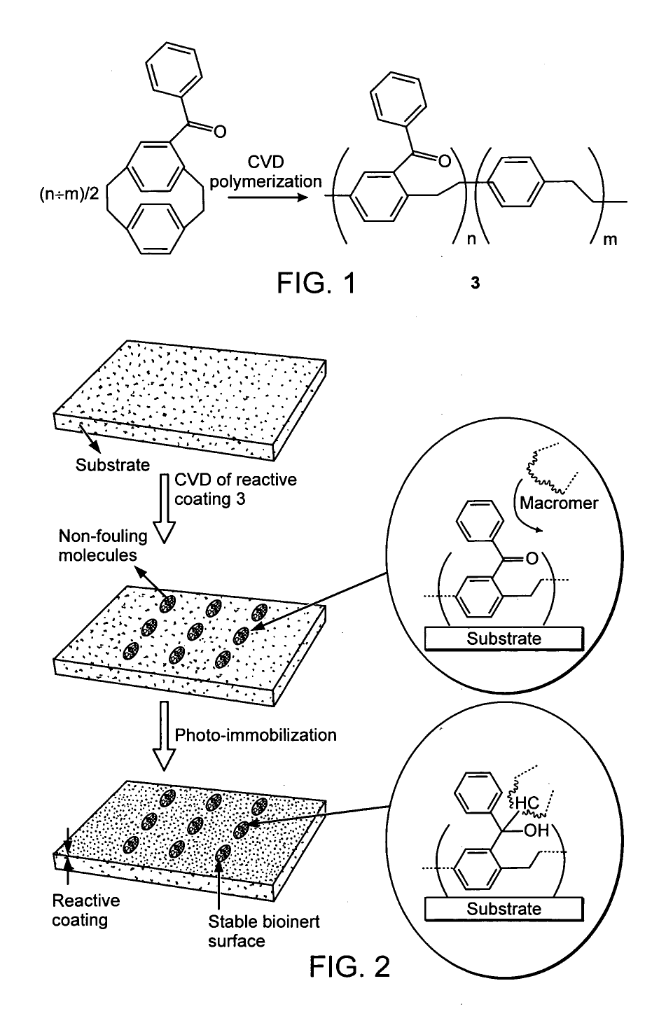

### DETAILED DESCRIPTION OF THE INVENTION

For CVD polymerization, carefully purified 4-benzoyl[2.2]paracyclophane (dimer) was evaporated under a reduced pressure and at a elevated temperatures. Prior to deposition, 4-benzoyl[2.2]paracyclophane was transferred to the pyrolysis zone, which was heated above 600° C., in same cases above 750 C, to ensure cleavage of the C—C bonds resulting in the corresponding quinodimethanes (monomers). In the last step, monomers were adsorbed on the substrate at temperatures around 5° C. and spontaneously polymerized. CVD polymerization delivered transparent and topologically uniform polymer films of thicknesses between 40 and 200 nm. The film thickness was determined by the amount of 4-benzoyl[2.2]paracyclophane used for polymerization. The elemental composition of the photodefinable polymer coating was determined by X-ray photoelectron spectroscopy (XPS) to be in good accordance with the theoretical composition. Decomposition of the benzoyl group was negligible. The IR spectrum of the photodefinable polymer coating confirmed the presence of the intact carbonyl bond as indicated by characteristic signals at wavelengths of 1665 and 1600 cm⁻¹. Polymer 3 was chemically stable under ambient conditions for several weeks as determined by IR spectroscopy. Similar to other functionalized poly-p-xylylenes, the photodefinable polymer coating showed good adhesion on various substrates, such as poly(dimethylsiloxane) (PDMS), poly(tetra-fluoroethylene), gold, glass, or silicon.

The photodefinable polymer coating is insoluble in ethanol or aqueous solutions. Incubation of a gold substrate coated with the photodefinable polymer coating in an aqueous phosphate buffered saline (PBS) buffer (pH 7.4) for 7 d at room temperature did not affect its mechanical stability. Due to its structural analogy to benzophenone, the polymer's aromatic keto group is photochemcially activated at wavelengths around 340 nm and spontaneously reacts with surrounding molecules via C—H abstraction. Therefore, the photodefinable polymer coating may have broad technical implications for confinement of secondary polymers.

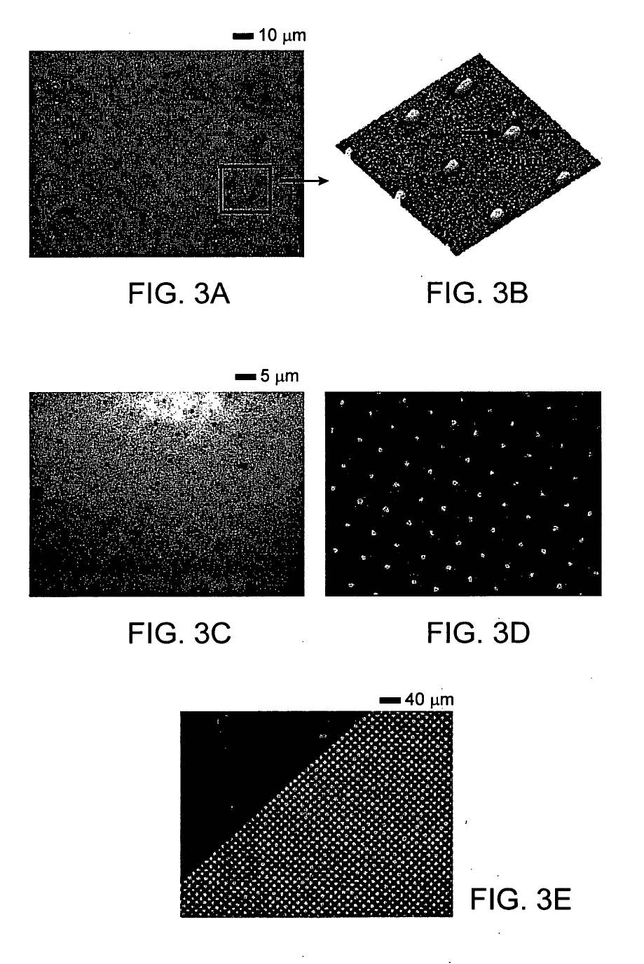

When microfabricating secondary polymer elements, one of the most challenging steps is the stable surface confinement of the secondary polymer. Stability of secondary polymer features was tested by dipping a patterned sample in PBS solutions and examining the structure by optical microscopy. Control experiments on glass substrates have shown that isolated PEG-DMA secondary polymer islands become unstable in PBS when the diameter is less than about 50 μm. As pattern size decreases and the surface-volume ratio increases, volume changes in the gel due to water-induce swelling are exhibited. Interconnected structures were more stable than isolated ones of the same size. FIG. 3 shows typical island structures that were fabricated on reactive coatings 3 using a 10 μm positive stamp. Both optical, FIG. 3a and atomic force microscopy (AFM) FIG. 3b, images indicate well-defined islands of 2-3 μm over large areas. Without the support of the photodefinable polymer coating, secondary polymer island are extremely unstable at this size range, such that they immediately delaminate from the surface upon hydration in PBS solution. Secondary polymer elements that were photochemically confined to the photodefinable polymer coating swelled less than 10% with respect to the initial diameter showing excellent stability in PBS solution for up to 72 h. In addition, the secondary polymers did not show an increase in size at the base of the structures because they were fixed to the substrate surface. To evaluate the potential applications of the stabilized secondary polymers, fluorescein-conjugated streptavidin was attached to the secondary polymer. A mixture of NHS-PEG-acrylate and PEG-dimethacrylate (50:50) in methanol (20 wt.-%) to introduce functional groups (N-hydroxysuccinimide, NHS) and allowed the resulting secondary polymers to react with an amino-functionalized biotin derivative. Biotin-based ligands were chosen since biotin is a prototype of a small ligand and interacts with streptavidin.

The structures are more resistant to water, and are vulnerable to water-induced attack at longer dipping times. Without the photodefinable polymer, delamination typically started from the edge region and advanced to the inner part of the pattern, which led to significant peel-off within a week. In parallel, the secondary polymer morphology drastically roughened through two mechanisms: water penetration into the surface and water induced swelling. For example, water penetrated deeply into the thin regions of the meniscus for a pattern size of less than 5 μm and relatively flat and thick secondary polymer films as shown in FIG. 2. In contrast, the introduction of an approximately 40 nm thick film of reactive coating 3 led to stable interconnected structures and completely suppressed delamination for up to several weeks.

In summary, the invention is directed to a novel, polymer using CVD polymerization that can act as a photodefinable reactive coating. It has a potential as a convenient and flexible interface for confinement of secondary polymers often required in biomedical coating processes. The proposed concept combines the advantages of a vapor-deposited polymer with the ability to conduct photochemical immobilation chemistry. The chemical nature of reactive coating provides unique opportunities for microfabrication of cross-linked polymers, as demonstrated for PEG secondary polymers. In additions, there are applications of photodefinable reactive coatings for fabrication of non-fouling surfaces in microfluidic devices, creation of cell patterns or as generic method to immobilize biomolecules.

The invention is applicable to a wide range of substrate materials, including stents, grafts, shunts, pacemakers, artificial heart valves, etc. The first step is the immobilization of the reactive coating with ensures strong adhesion to the device and the capability to support photoreactions. The photoreaction step can be executed in the presence of a secondary polymer, biomolecules, buffer, or combinations thereof. The photoreaction can also be conducted in form of a photopatterning step. In this case, standard methods for photopatterning, as known to an expert skilled in the art, will be applicable. However, the photopatterning is optional and the photoreaction step can also be applied to the entire device or preferred areas thereof, without the use of patterning techniques. The thickness of the secondary polymer is flexible. Typical thicknesses are between 100 nm and several millimeters. However, these limits do not establish fundamental barriers and both, thinner and thicker films can be fabricated as needed. The secondary polymer can act as a carrier for functional moieties, such as drugs, biological cues, or cells including genetically modified cells or stem cells.

The foregoing description has been limited to a few embodiments of the invention. It will be apparent, however, that variations and modifications can be made to the invention, with the attainment of some or all of the advantages. Therefore, it is the object of the claims to cover all such variations and modifications as come within the true spirit and scope of the invention.

1. A photodefinable polymer having the following structure: 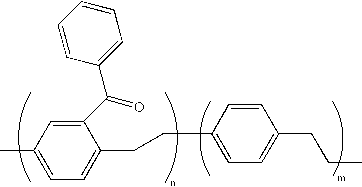
where m is 0 to 10000 and n is 0 to 10000.

2. A photodefinable polymer coating, said coating prepared from the following compound: 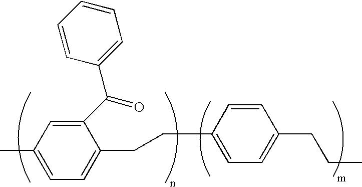
where m is 0 to 10000 and n is 0 to 10000.

3. A biomedical device having a coating on at least a portion thereof, the coating having a formulation as claimed in claim 2.

4. The polymer of claim 1, wherein the polymer is an adhesion promotor for a drug delivery system.

5. The polymer of claim 4, wherein the polymer is coated onto a substrate such that it is a reactive coating for UV crosslinking.

6. The polymeric coating of claim 2, wherein the polymeric coating provides an interface for an implant.

7. The polymeric coating of claim 6, wherein the polymeric coating may be modified with a secondary polymer or polymer combination.

8. The polymeric coating of claim 7, wherein the secondary polymer is capable of eluding drugs or encapsulating cells.

9. A polymer coated stent, wherein at least parts of the stent device are coated with the polymer coating according to claim 2.

10. The stent according to claim 9, where the stent is modified with a secondary drug eluding polymer through photoreaction.

11. The biomedical device of claim 3 wherein the device may be selected from a pacemaker, shunt, catheter, artificial heart valves, and embolization coils.

12. The photodefinable polymer coating according to claim 8, wherein the cells are genetically modified.

13. The photodefinable polymer coating according to claim 8, wherein the resulting device is used for gene therapy.

14. A chemical vapor deposition process, said process includes coating a substrate with the photefinable coating according to claim 2.

15. The chemical vapor deposition process of claim 14, wherein [2.2]paracyclophanes are polymerized during the chemical vapor deposition process.

16. The chemical vapor deposition process of claim 14, wherein photolithography is used to create immobilization pattern on a substrate.

17. The chemical vapor deposition of claim 2, wherein a coating is deposited onto a substrate, said process including:
providing purified 4-benzoyl[2.2]paracyclophane; sublimating the 4-benzoyl[2.2]paracyclophane under a reduced pressure of less than 100 Pa;
heating the sublimated 4-benzoyl[2.2]paracyclophane to approximately 550° C.-900° C. to cleave C—C bonds to produce monomers;
polymerizing the monomers which are absorbed on the substrate at a temperature below 150° C. to produce a topologically uniform polymer coating.

18. The chemical vapor deposition process of claim 14, wherein the polymer coating is transparent.

19. The chemical vapor deposition process of claim 14, wherein the polymeric coating has a thickness between 40 and 2000 nm.

20. The chemical vapor deposition process as claimed in claim 14, further including masking a surface of the substrate to produce a patterned coating having defined areas, each area having different functional groups.

21. The chemical vapor deposition process as claimed in claim 14 further including a plasma treatment of the substrate prior to the chemical vapor deposition process.

22. The chemical vapor deposition process as claimed in claim 14, wherein a gradient of reactivity is formed.
